(19) United States
(12) Patent Application Publication
(10) Pub. No.: US 2004/0026230 A1
(43) Pub. Date: Feb. 12, 2004

(54) Process for preparing intermediates of hiv protease inhibitors

(76) Inventors: Roman Davis, Durham, NC (US); Thomas Claiborne Lovelace, Durham, NC (US)

Correspondence Address: DAVID J LEVY, CORPORATE INTELLECTUAL PROPERTY, GLAXOSMITHKLINE, FIVE MOORE DR., PO BOX 13398, RESEARCH TRIANGLE PARK, NC, 27709-3398, US

(21) Appl. No.: 10/399,809

(22) PCT Filed: Oct. 22, 2001
(86) PCT No.: PCT/US01/51428

(51) Int. Cl. C07D 487/02

(52) U.S. Cl. 204/157.7; 204/157.71

(57) ABSTRACT

The invention describes a method for effecting intramolecular cyclization reactions using light, in the absence of radical initiators, to provide useful polycyclic compounds.

### BACKGROUND OF THE INVENTION

The human immunodeficiency virus (“HIV”) is the causative agent for acquired immunodeficiency syndrome (“AIDS”), a disease characterized by the destruction of the immune system, particularly of CD⁴⁺T-cells, with attendant susceptibility to opportunistic infections, and its precursor AIDS-related complex (“ARC”), a syndrome characterized by symptoms such as persistent generalized lymphadenopathy, fever and weight loss.

Among the drugs currently used to treat HIV infections in humans are those that inhibit the HIV aspartyl protease enzyme. Drugs that are used as protease inhibitors are, in general, chemically complex and are difficult to prepare in a cost-effective and efficient manner. As a result of the inherent complexity of these molecules, new and more efficient methods for their preparation are of value.

The synthesis of hexahydrofuro[2,3-b]furan-3-ol, an intermediate in the synthesis of HIV aspartyl protease inhibitors, was described by Ghosh, et. al. (J. Med. Chem. 1996, 39(17), p. 3278). A key step in the preparation of the hexahydrofuro[2,3-b]furan ring system is the cyclization of a 2-(2-propynyloxy)tetrahydrofuranyl derivative under radical cyclization conditions. For example, Ghosh et. al. reported that 3-iodo-2-(2propynyloxy)tetrahydrofuran could be cyclized to the desired 3-methylene hexahydrofuro[2,3-b]furan derivative using stoichiometric amounts of compounds capable of acting as radical initiators, such as a mixture of sodium borohydride and cobaloxime. Alternatively, the same cyclization reaction can be effected using a stoichiometric amount of a trialkyltin hydride, such as tributyltin hydride. There are disadvantages to such methods for the synthesis of pharmaceutical intermediates, for example, toxicity of trace amounts of metals such as cobalt or tin. As a result of toxicity concerns, we developed a new process for the preparation of the hexahydrofuro[2,3-b]furan ring system that avoids the use of toxic metals in the key cyclization step.

The cyclization of O-alkenyl aryl radicals to provide dihydrobenzofurans has been described by Beckwith, et. al. (J. Chem. Soc., Chem. Comm. 1981, p. 136). In these reactions, 2halo-O-allylic phenolic precursor was prepared and the required aryl radical intermediate was generated using tributyltin hydride.

The radical cyclization of O-allylic-2-halo sugar derivatives to afford alpha-C(2)-branched sugars has been described by Mesmaeker, et al. (Synlett 1990, p. 201). This method utilizes radical initiating compounds, such as a combination of AIBN and tributyltin hydride, in addition to the use of light to effect the desired cyclizations.

### SUMMARY OF THE INVENTION

The invention comprises a method for effecting intramolecular cyclization reactions using light, in the absence of radical initiators, to provide useful polycyclic compounds. The present invention further provides a method of preparation of an intermediate useful in the synthesis of compounds that function as inhibitors of the aspartyl protease enzyme of human immunodeficiency virus (HIV). The present method is characterized by the use of light to effect cyclization of a hexahydrofuro[2,3-b]furan ring system from a 2-(2propynyloxy)tetrahydrofuranyl derivative without the use of a stoichiometric amount of a radical initiator. The hexahydrofuro[2,3-b]furan derivative may be transformed through a series of further reactions to produce hexahydrofuro[2,3-b]furan-3-ol, an intermediate in the synthesis of compounds that are effective as inhibitors of HIV aspartyl protease.

### DETAILED DESCRIPTION OF THE INVENTION

The present method involves the use of light to effect the intramolecular cyclization of an appropriately substituted organic halo compound containing a pendant olefinic or alkynyl substituent. The invention is summarized in Schemes I, II, and IV wherein A, which may be the same or different, is independently selected from the group consisting of —CH₂—, —CHR¹⁰—, —CR¹⁰R¹¹—, —O—, —NH—, —NR¹⁰—, —S—; wherein R¹⁰ and R¹¹, which may be the same or different, are selected from the group consisting of hydrogen, C₆₋₁₄aryl, and C₁₋₆alkyl; R¹ is selected from the group consisting of hydrogen, C₁₋₆alkyl, C₆₋₁₄aryl, C₁₋₆alkylheterocycle, and heterocycle; R² is selected from the group consisting of hydrogen, C₆₋₁₄aryl, C₁₋₆alkyl, C₆₋₁₄aryl, C₁₋₆alkylheterocycle, and heterocycle; R³ is selected from the group consisting of hydrogen, C₆₋₁₄aryl, C₁₋₆alkyl, C₁₋₆alkylheterocycle, and heterocycle; R⁴ is selected from the group consisting of hydrogen, C₆₋₁₄aryl, C₁₋₆alkyl, C₁₋₆-alkylheterocycle, and heterocycle; R⁵ is selected from the group consisting of hydrogen, C₆₋₁₄aryl, C₁₋₆alkyl, C₁₋₆alkylheterocycle, heterocycle, —OR¹², and —CH₂OR¹², wherein R¹² is selected from the group consisting of C₁₋₆alkyl and —C(O)R¹⁰; R⁶ is selected from the group consisting of hydrogen, C₆₋₁₄aryl, C₁₋₆alkyl, C₁₋₆alkylheterocycle, heterocycle, —OR¹², and —CH₂OR¹², wherein R¹² is selected from the group consisting of C₁₋₆alkyl and —C(O)R¹⁰; R⁷, R⁸ and R⁹, which may be the same or different are selected from the group consisting of hydrogen, C₆₋₁₄aryl, C₁₋₆alkyl, C₁₋₆alkylheterocycle, and heterocycle; X is halogen; and n=1-4.

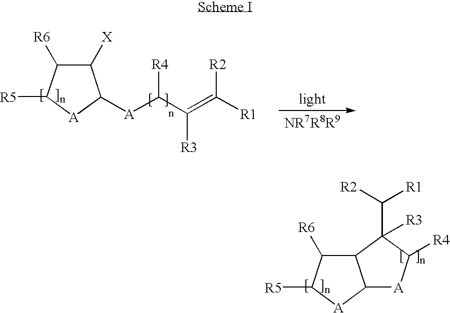

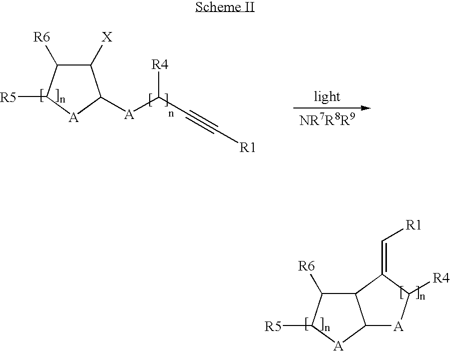

The present invention features a process for the preparation of a compound having the formula

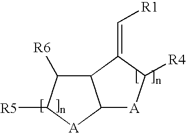

wherein,

A, which may be the same or different, is independently selected from the group consisting of —CH₂—, —CHR¹⁰—, —CR¹⁰R¹¹—, —O—, —NH—, —NR¹⁰ —, and —S—, wherein R¹⁰ and R¹¹, which may be the same or different, are selected from the group consisting of hydrogen, C₆₋₁₄aryl, and C₁₋₆-alkyl;

R¹ is selected from the group consisting of hydrogen, C₁₋₆alkyl, C₆₋₁₄aryl, C₁₋₆alkylheterocycle, and heterocycle;

R⁴ is selected from the group consisting of hydrogen, C₆₋₁₄aryl, C₁₋₆alkyl, C₁₋₆alkylheterocycle, and heterocycle;

R⁵ is selected from the group consisting of hydrogen, C₆₋₁₄aryl, C₁₋₆alkyl, C₁₋₆alkylheterocycle, heterocycle, —OR¹², and —CH₂OR¹², wherein R¹² is selected from the group consisting of C₁₋₆alkyl and —C(O)R¹⁰;

R⁶ is selected from the group consisting of hydrogen, C₆₋₁₄aryl, C₁₋₆alkyl, C₁₋₆alkylheterocycle, heterocycle, —OR¹², and —CH₂OR¹²; and

n=1-4

said process comprising:

exposing a compound having the formula

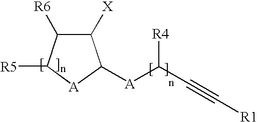

wherein R¹ to R⁶ are as hereinbefore defined to light with a wavelength of 200 to 400 nanometers in the presence of a compound of formula NR⁷R⁸R⁹, wherein R⁷, R⁸ and R⁹, are independently selected from the group consisting of hydrogen, C₆₋₁₄aryl, C₁₋₆alkyl, C₁₋₆alkylheterocycle, and heterocycle.

The present invention further features a process for the preparation of 3methylenehexahydrofuro[2,3-b]furan in the absence of radical initiators comprising exposing a 3-halo-2-(2-propynyloxy)tetrahydrofuranyl derivative to light with a wavelength from 200 to 400 nanometers, in the presence of a solvent containing a compound of formula NR⁷R⁸R⁹, wherein R⁷, R⁸ and R⁹, are independently selected from the group consisting of hydrogen, C₆₋₁₄aryl, C₁₋₆alkyl, C₁₋₆alkylheterocycle, and heterocycle, thereby cyclizing the 3-halo-2-(2propynyloxy)tetrahydrofuranyl derivative to form 3-methylenehexahydrofuro[2,3-b]furan.

The present invention also features a process for the preparation of hexahydrofuro[2,3b]furan-3-ol consisting of:

a) exposing a 3-halo-2-(2-propynyloxy)tetrahydrofuranyl derivative to light with a wavelength from 200-400 nanometers in the presence of a solvent containing a compound of formula NR⁷R⁸R⁹, wherein R⁷, R⁸ and R⁹, are independently selected from the group consisting of hydrogen, C₆₋₁₄aryl, C₁₋₆alkyl, C₁₋₆alkylheterocycle, and heterocycle, thereby cyclizing the 3-halo-2-(2-propynyloxy)tetrahydrofuranyl derivative to form 3methylenehexahydrofuro[2,3-b]furan;

b) oxidizing said 3-methylenehexahydrofuro[2,3-b]furan to produce tetrahydrofuro[2,3b]furan-3(2M-one; and

c) reducing said tetrahydrofuro[2,3-b]furan-3(2M-one to yield hexahydrofuro[2,3-b]furan3-ol.

The present invention includes a process as described above wherein the 3-halo-2-(2propynyloxy)tetrahydrofuranyl derivative may be 3-iodo-2-(2-propynyloxy)tetrahydrofuran, 3-bromo-2-(2-propynyloxy)tetrahydrofuran, or 3-chloro-2-(2-propynyloxy)tetrahydrofuran, the light is at a wavelength of 254 nanometers, and the compound of formula NR⁷ R⁸ R⁹ is triethylamine.

The processes of the present invention involve the initial preparation of a suitable substrate for the intramolecular photocyclization reaction. These substrates can be prepared by a number of methods known to one skilled in the art. For example, preparation of a 3-halo-2-(2-propynyloxy)tetrahydrofuranyl derivative is effected by reaction of 2,3-dihydrofuran with 2-propyn-1-ol in the presence of a suitable activating agent, to provide a 3-halo-2-(2-propynyloxy)tetrahydrofuran derivative. For example, such 3-halo-2-(2-propynyloxy)tetrahydrofuran derivatives can be 3-iodo-2-(2-propynyloxy)tetrahydrofuran, 3bromo-2-(2-propynyloxy)tetrahydrofuran or 3-chloro-2-(2-propynyloxy)tetrahydrofuran.

This reaction can be effected using an agent capable of activating the 2,3-dihydrofuran ring to nucleophilic addition by the alcoholic portion of a 2-propyn-1-ol derivative. For example, the reaction can be performed using N-bromosuccinimide (NBS) or N-iodosuccinimide (NIS), in a non-nucleophilic solvent, such as dichloromethane, and at temperatures from −10° C. to 25° C., preferably 0° C., followed by warming to 25° C. For example, 2,3-dihydrofuran was allowed to react with 2-propyn-1-ol in dichloromethane and in the presence of NBS to provide 3-bromo-2-(2-propynyloxy)tetrahydrofuran (Scheme III).

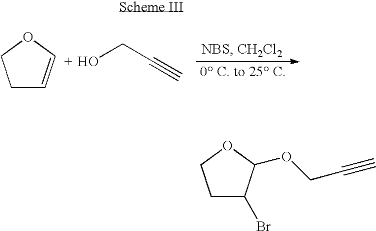

The 3-halo-2-(2-propynyloxy)tetrahydrofuran derivative, such as 3-iodo-2-(2propynyloxy)tetrahydrofuran, 3-bromo-2-(2-propynyloxy)tetrahydrofuran or 3-chloro-2-(2propynyloxy)tetrahydrofuran, may be cyclized using light, in the presence of a trialkyl amine and in the presence of a suitable solvent This reaction may be performed using a light source that is sufficient to cause homolytic cleavage of the carbon-halogen bond in the 3-halo-2-(2-propynyloxy)tetrahydrofuran derivative. For example, the light source used can provide ultraviolet light of sufficient intensity to cause the desired homolytic cleavage of the carbonhalogen bond. Preferably, a light source is used that provides light with a wavelength of 254 nanometers, produced by low-pressure mercury lamps. These reactions may be performed in a suitable solvent, one that is sufficiently stable under the photolytic conditions. For example, the reaction may be performed in a polar solvent such as acetonitrile. It has also been found that the cyclization reaction may be performed in the presence of a suitable trialkylamine of formula NR⁷R⁸R⁹, wherein R⁷, R⁸, and R⁹ are independently selected from the group consisting of hydrogen, C₁₋₆alkyl, C₆₋₁₄aryl, heterocycle, and C₁₋₆alkylheterocycle, preferably C₁₋₆alkyl. Preferably the amine is triethylamine. We have discovered that the reaction may be advantageously performed in the presence of water. The amount of water that may be used will vary depending on both the solvent and the trialkylamine chosen.

The reaction may be performed in any suitable reaction vessel that will allow the passage of a sufficient amount of light in the preferred wavelength. The reaction may be advantageously performed in a vessel that is suitable as a flow-cell reactor.

For example, the 3-bromo-2-(2-propynyloxy)tetrahydrofuran may be photolyzed using light at a wavelength of 254 nanometers, in a 3:7:5 ratio of acetonitrile, triethylamine and water at 20° C. for 15-20 hours, to afford 3-methylenehexahydrofuro[2,3-b]furan (Scheme IV).

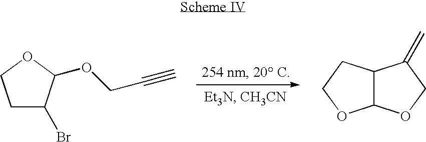

The intermediate methylenehexahydrofuro[2,3-b]furan may then be oxidized to produce the desired tetrahydrofuro[2,3-b]furan-3(2H)-one. The oxidation may be performed using a variety of methods well known to those skilled in the art. For example, the olefin can be allowed to react with osmium (IV) oxide, followed by treatment with an agent capable of cleaving the resulting diol, sodium periodate for example. Alternatively, the olefin can be treated with ozone in a suitable solvent, followed by the addition of an agent capable of cleaving the resulting ozonide. These reactions are typically performed in an organic solvent that is stable to the reaction conditions, dichloromethane for example, and at temperatures from −75° C. to 25° C., advantageously from −30° to 20° C. For example, methylenehexahydrofuro[2,3-b]furan may be allowed to react with ozone in methylene chloride at −30° C., followed by warming to −20° C. and the addition of triethylamine, to provide tetrahydrofuro[2,3-b]furan-3(2M-one (Scheme V).

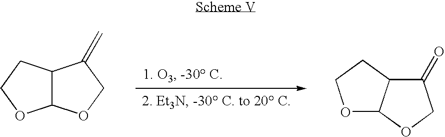

The intermediate tetrahydrofuro[2,3-b]furan-3(2M-one may then be reduced to yield hexahydrofuro[2,3-b]furan-3-ol. The reduction may be performed using an appropriate reducing agent, sodium borohyride or diisobutylaluminum hydride, or more preferably lithium aluminum hydride, in the presence of an aprotic, organic solvent, preferably dichloromethane, and at a temperature from 0° C. to 40° C., preferably in the range from 20-30° C. The choice of an appropriate reducing agent will depend on factors known to those skilled in the art and include the properties of the particular compound being reduced and those of the solvent in which the reaction is being performed. For example, tetrahydrofuro[2,3-b]furan-3(2H)-one may be allowed to react with lithium aluminum hydride in dichloromethane as solvent and at a temperature range of 20-30° C. to yield hexahydrofuro[2,3-b]furan-3-ol as a racemic mixture (Scheme VI).

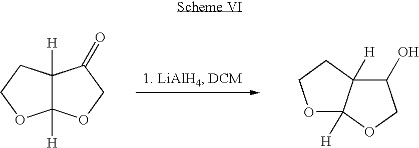

Enantioenriched hexahydrofuro[2,3-b]furan-3-ol may also be obtained by the use of so-called “chiral reducing agents.” These agents are capable of reducing ketones and aldehydes in an enantioselective fashion to provide enantioenriched alcohols. The reactions may be performed with stoichiometric as well as catalytic chiral reducing agents using conditions known to those skilled in the art. For example, see Ernest L Eliel, “Stereochemistry of Organic compounds,” John Wiley Et Sons, Inc., 1994, p. 941.

Alternatively, a racemic mixture of hexahydrofuro[2,3-b]furan-3-ol may be resolved to provide an enantioenriched mixture of each enantiomer. There are several different methods to accomplish this type of resolution known to those skilled in the art.

First, a racemic mixture of hexahydrofuro[2,3-b]furan-3-ol may be resolved by converting the mixture of enantiomers into a mixture of diastereomers, followed by traditional methods of separation, such as silica chromatography. In this type of resolution, the racemic alcohol may be allowed to react with a chiral nonracemic compound (the resolving agent) resulting in the formation of a diastereomeric mixture. Typically, the chiral nonracemic compound is either an acid chloride or a chloroformate, resulting in the formation of a diastereomeric mixture of esters or ureas, respectively. The choice of the chiral nonracemic resolving agent will depend on factors known to those skilled in the art. For example, see Eliel, et. al., p. 322.

Next, the racemic alcohol may be allowed to react with a lipase enzyme capable of converting one enantiomer of the alcohol into an ester. The ester and the remaining alcohol may then be separated by methods known to those skilled in the art. See Eliel, et al., p. 413.

Lastly, the racemic alcohol may be separated into two enantioenriched mixtures by the use of an esterase. These reactions typically consist of first converting the racemic alcohol to an appropriate ester, such as the corresponding acetate. The conversion of the alcohol to the corresponding ester can be accomplished by reaction of the alcohol with an appropriate agent, an acid chloride or anhydride for example. These reactions are typically performed in an aprotic solvent, tetrahydrofuran for example, and in the presence of a compound capable of acting as a base, sodium carbonate for example. In addition, a compound capable of acting as a catalyst may be advantageously used, for example 4-N,N-dimethylaminopyridine. The racemic mixture of esters may then be allowed to react with an appropriate esterase enzyme under conditions which allow for reaction of predominantly one racemate of the ester to provide a mixture of an alcohol of predominantly one enantiomer and the remaining ester, consisting of predominantly the other enantiomer. The mixture of alcohol and ester may then be separated using methods known to those skilled in the art, silica gel chromatography for example. The choice of an appropriate esterase enzyme, as well as appropriate reaction conditions will depend on a number of factors known to those skilled in the art Eliel, et al., p. 409. For example, racemic hexahydrofuro[2,3-b]furan-3-ol may be allowed to react with acetic anhydride in a mixture of tetrahydrofuran and water, and in the presence of sodium carbonate and 4-N,N-dimethylaminopyridine to yield hexahydrofuro[2,3-b]furan-3-yl acetate. The resulting acetate may then be allowed to react with PS-800 in a buffered mixture of sodium hydrogen phosphate while the pH is kept between 6.2 and 7.2 with the addition of 15% aqueous sodium hydroxide as needed to yield a mixture of (3R,3aS,6aR)-hexahydrofuro[2,3-b]furan-3-yl acetate and (3S,3aR,6aS)-hexahydrofuro[2,3b]furan-3-ol (Scheme VII).

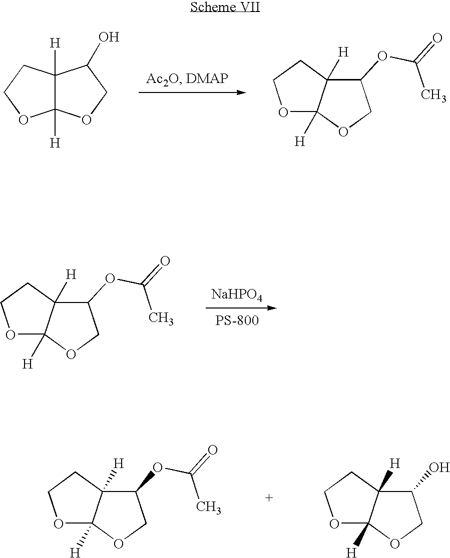

The term “alkyl”, alone or in combination with any other term, refers to a straight-chain or branched-chain saturated aliphatic hydrocarbon radical containing the specified number of carbon atoms. Examples of alkyl radicals include, but are not limited to, methyl, ethyl, n-propyl, isopropyl, n-butyl, isobutyl, sec-butyl, tert-butyl, pentyl, isoamyl, n-hexyl and the like.

The term “aryl,” alone or in combination with any other term, refers to a carbocyclic aromatic radical (such as phenyl or naphthyl) containing the specified number of carbon atoms, preferably from 6-14 carbon atoms, and more preferably from 6-10 carbon atoms. Examples of aryl radicals include, but are not limited to phenyl, naphthyl, indenyl, indanyl, azulenyl, fluorenyl, anthracenyl and the like. In addition, the aryl ring may be optionally substituted with one or more groups independently selected from the group consisting of halogen, C₁₋₆alkyl, —CF₃, heterocycle, —OCH₃, aryl, C₁-alkylaryl, and C₁₋₆-alkylheterocycle.

The term “halogen” refers to a radical of chlorine, bromine or iodine.

The term “heterocycle” or “heterocyclic” as used herein, refers to a 3-to 7-membered monocyclic heterocyclic ring or 8- to 11-membered bicyclic heterocyclic ring which is either saturated, partially saturated or unsaturated, and which may be optionally benzofused if monocyclic. Each heterocycle consists of one or more carbon atoms and from one to four heteroatoms selected from the group consisting of N, O and S, and wherein the nitrogen and sulfur heteroatoms may optionally be oxidized, and including any bicyclic group in which any of the above-defined heterocyclic rings is fused to a benzene ring. The heterocyclic ring may be attached at any carbon or heteroatom, which results in the creation of a stable structure. Preferred heterocycles include 5-7 membered monocyclic heterocycles and 8-10 membered bicyclic heterocycles. Examples of such groups include imidazolyl, imidazolinoyl, imidazolidinyl, quinolyl, isoqinolyl, indolyl, indazolyl, indazolinolyl, perhydropyridazyl, pyridazyl, pyridyl, pyrrolyl, pyrrolinyl, pyrrolidinyl, pyrazolyl, pyrazinyl, quinoxolyl, piperidinyl, pyranyl, pyrazolinyl, piperazinyl, pyrimidinyl, pyridazinyl, morpholinyl, thiamorpholinyl, furyl, thienyl, triazolyl, thiazolyl, carbolinyl, tetrazolyl, thiazolidinyl, benzofuranoyl, thiamorpholinyl sulfone, oxazolyl, benzoxazolyl, oxopiperidinyl, oxopyrrolidinyl, oxoazepinyl, azepinyl, isoxozolyl, isothiazolyl, furazanyl, tetrahydropyranyl, tetrahydrofuranyl, thiazolyl, thiadiazoyl, dioxolyl, dioxinyl, oxathiolyl, benzodioxolyl, dithiolyl, thiophenyl, tetrahydrothiophenyl, sulfolanyl, dioxanyl, dioxolanyl, tetahydrofurodihydrofuranyl, tetrahydropyra nodihydrofuranyl, dihydropyranyl, tetradyrofurofuranyl and tetrahydropyranofuranyl.

The term “flow-cell reactor” refers to a vessel that is suitable for use in chemical reactions. In general, a vessel suitable for use as a flow-cell reactor for photolytic chemical reactions comprises a hollow container with a smooth, reflective interior, constructed of a suitable material, preferably stainless steel, an inlet and outlet suitable for the introduction and removal of a chemical reaction mixture, and a light source capable of providing light in the range of 200-400 nanometers, preferably 254 nanometers.

The following examples are for the purpose of illustration only and are not to be construed as limiting the scope of the invention in any way.

### EXAMPLE 1

A. 3-Bromo-2-(2-propynyloxy)tetrahydrofuran.

A reactor was charged with N-bromosuccinimide (NBS, 1.05 eq., 2.67 wt), followed by methylene chloride (10 vol). The resulting slurry was cooled to 0° C., and a mixture of 2,3dihydrofuran (1 eq., 1.0 wt), and propargyl alcohol (1.5 eq., 1.2 wt) was added over 40 min. The resulting clear solution was stirred at 0° C. for 1 h, heated to 25° C. over 30 min and held at that temperature overnight. The solution was then washed with water (1×10 vol), 25% sodium meta-bisulfite (2×5 vol) and saturated sodium bicarbonate (25 vol). The resulting solution was then concentrated under vacuum to an oil. Acetonitrile (1 vol) was added, and the solution concentrated to an oil under vacuum to provide 3-bromo-2-(2-propynyloxy)tetrahydrofuran. ¹H NMR of the title compound was identical to that found in the literature (Ghosh, et al., J. Med. Chem. 1996, 39(17), p. 3278).

B. 3-Methylenehexahydrofuro[2,3-b]furan

3-bromo-2-(2-propynyloxy)tetrahydrofuran (1 eq., 1 wt) was dissolved in acetonitrile (3 vol), triethylamine (10.3 eq., 7 vol) and water (5 vol) in a jacketed reactor. The solution was stirred at 20° C. and was circulated through a stainless photoreactor equipped with ten 13.8W low-pressure, mercury lamps (254 nm output) for 15-20 h. After the appropriate reaction time, the mixture was concentrated to an oil. ¹H NMR of the title compound was identical to that found in the literature (Ghosh, et. al., J. Med. Chem. 1996, 39(17), p. 3278). Reaction progress was followed using a gas chromatograph under the following conditions:

Column: DB-624 30 m×0.53 mm column with a 3 micron film thickness;

Carrier gas: He at 5 mL/min;

Injector temperature: 250° C.;

Detector type and temperature: FID, 300° C.;

Initial oven temperature: 100° C.;

Temperature ramp: 20 degrees/min to 250° C., followed by a 7.5 min hold.

Retention time of 3-methylenehexahydrofuro[2,3-b]furan =4.9 min.

C. Tetrahydrofuro[2,3-b]furan-3(2M-one

A reactor was charged with 3-methylenehexahydrofuro[2,3-b]furan (1 eq., 1.0 wt), and methylene chloride (10 vol). The solution was stirred and cooled to −30° C. Ozone was introduced through a subsurface addition line while the temperature was kept at −30+/−5° C. When the solution turned blue, it was purged with nitrogen and triethylamine (2.0 eq) was slowly added, keeping the temperature between −30 and −20° C. After the addition was complete, the solution was allowed to warm to 20° C. and was allowed to stir overnight After stirring overnight, 3 N HCl and 5% brine solution (3.2 vol) were added at such a rate as to keep the temperature of the reaction mixture below 30° C. The layers were then separated and the organic layer was washed with 5% brine solution, and then concentrated under vacuum (15 mbar) to provide tetrahydrofuro[2,3-b]furan-3(2m-one as an oil. ¹H NMR of the title compound was identical to that found in the literature (Ghosh, et. al., J. Med. Chem. 1996, 39(17), p. 3278).

D. Hexahydrofuro[2,3-b]furan-3-ol

A reactor was charged with tetrahydrofuro[2,3-b]furan-3(2hM-one (1.0 eq., 1.0 wt) and methylene chloride (5 vol). Lithium aluminum hydride (1 M in tetrahydrofuran, 0.45 eq., 5.9 vol) was added slowly in order to keep the reaction temperature below 30° C. After the addition was complete, the reaction was stirred an additional 30 min and then was cooled in an ice bath. The following were successively added at a rate such that the temperature of the reaction mixture remained below 20° C.: 25% water in THF (4 vol v. LAH solid wt), 15% w/v sodium hydroxide (3 vols v. LAH solid wt), and water (1 vol v. LAH solid wt). Celite was added immediately after the addition of water was complete and the resulting slurry was stirred for 1 h. The slurry was then filtered through a coarse fritted funnel, and the filter cake was washed with THF (2 vol). The filtrate and washings were combined and were used in the next step without further purification or manipulation. ¹H NMR of the title compound was identical to that found in the literature (Ghosh, et. al., J. Med. Chem. 1996, 39(17), p. 3278).

E. Hexahydrofuro[2,3-b]furan-3-yl acetate

A reactor was charged with sodium carbonate (2.5 eq., 2.0 wt) the filtrate from step D above, and 4,4-N,N-dimethylaminopyridine (0.05 eq., 0.04 wt). The resulting mixture was cooled in an ice bath and acetic anhydride (1.5 eq., 1.1 vol) was added at such a rate that the reaction mixture stayed below 10° C. The mixture was then allowed to warm to room temperature and stir overnight. The resulting slurry was filtered through a coarse fritted funnel and the filter cake was washed with methylene chloride (2 vol). The filtrate and washings were combined and were further extracted with 1 N HCl (1 vol). The mixture was then concentrated under vacuum to provide hexahydrofuro[2,3-b]furan-3-yl acetate as an oil. ¹H NMR of the title compound was identical to that found in the literature (Ghosh, et al., J. Med. Chem. 1996, 39(17), p. 3278).

F. (3R,3aS,6a R)-Hexahydrofuro[2,3-b]furan-3-yl acetate

A reactor was charged with 0.1 N NaHPO₄ (pH=7.0, 7.5 vol) and hexahydrofuro[2,3b]furan-3-yl acetate (1 eq., 1 wt). The pH of the solution was then adjusted to 7.0 by the addition of 15% sodium hydroxide and the solution was heated to 35+/−3° C. PS-800 (500 units/mmol) was then added and the pH was kept between 6.8 and 7.2 with the periodic addition of 15% sodium hydroxide. Reaction progress was followed by chiral gas chromatography until all of the undesired acetate had been hydrolyzed. Celite (0.5 wt) was then added, followed by methylene chloride (4.0 vol), and the resulting slurry was stirred for 15 min. The mixture was then filtered through a pad of celite, followed by several washes of the celite pad with methylene chloride. The organic layer was separated and the organic layer was washed with water (3×1 vol), 10% sodium chloride (2 vol) and then was concentrated under vacuum to provide (3R,3aS,6aR)-hexahydrofuro[2,3-b]furan-3-yl acetate as an oil. ¹H NMR of the title compound was identical to that found in the literature (Ghosh, et. al., J. Med. Chem. 1996, 39(17), p. 3278). Typical optical purity of the resulting (3R,3aS,6aR)hexahydrofuro[2,3-b]furan-3-yl acetate was >98% ee. Optical purity was determined using chiral GC under the following conditions:

Column: Astec Chiraldex Beta Cyclodextrin Trifluoroacetyl (B-TA) 20 m×0.25 mm;

Carrier gas: He @ 1 mL/min;

Make-up gas: He @ 30 mL/min

Detection: FID @ 300° C.

Injection:: 1 uL @ 250° C. (split)

Split flow: 100 mL/min

Total run time: 30 min

Temperature program: Isothermal @ 115° C.

Sample preparation: Approximately 25-50 mg sample (1-2 drops) in 10 mL acetonitrile. Inject 1 uL sample prep. The sample concentration may be adjusted as needed to give adequate sensitivity or to prevent column overloading.

Retention times:

(3S,3aR,6aS)-Hexahydrofuro[2,3-b]furan-3-yl acetate=11.43 min;

(3R,3aS,6aR)-Hexahydrofuro[2,3-b]furan-3-yl acetate=12.20 min.

G. (3R,3aS,6aR)-hexahydrofuro[2,3-b]furan-3-ol

A reactor was charged with (3R,3aS,6aR)-Hexahydrofuro[2,3-b]furan-3-yl acetate (1 eq., 1 wt), methanol (3 vol) and potassium carbonate (0.001 eq, 0.001 wt). The mixture was allowed to stir at rt for 18-20 h, after which time the reaction mixture was concentrated to afford (3R,3aS,6aR)-hexahydrofuro[2,3-b]furan-3-ol as an oil. ¹H NMR of the title compound was identical to that found in the literature (Ghosh, et. al., J. Med. Chem. 1996, 39(17), p. 3278). Reaction progress was followed using gas chromatography under the following conditions:

Column: DB-624, 30 m×0.53 mm×3 micron film thickness;

Carrier gas: He at 5 mL/min;

Makeup gas: He at 25 mL/min;

Detector: FID at 300° C.;

Initial oven temperature: 100° C. for 0 min;

Temperature ramp: 20° C./min, to 250° C., followed by a 7.5 min hold. Retention time of (3R,3aS,6aR)-hexahydrofuro[2,3-b]furan-3-ol=6.55 min.

We claim:

1. A process for the preparation of a compound having the formula 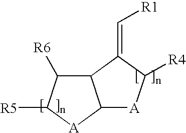
wherein,
A, which may be the same or different, is independently selected from the group consisting of —CH₂—, —CHR¹⁰—, —CR¹⁰R¹¹—, —O—, —NH—, —NR¹⁰O—, and —S—, wherein R¹⁰ and R¹¹, which may be the same or different, are selected from the group consisting of hydrogen, C₆₋₁₄aryl, and C₁₋₆alkyl;
R¹ is selected from the group consisting of hydrogen, C₁₋₆alkyl, C₆₋₁₄aryl, C₁₋₆alkylheterocycle, and heterocycle;
R⁴ is selected from the group consisting of hydrogen, C₆₋₁₄aryl, C₁₋₆alkyl, C₁₋₆alkylheterocycle, and heterocycle;
R⁵ is selected from the group consisting of hydrogen, C₆₋₁₄aryl, C₁₋₆alkyl, C₁₋₆alkylheterocycle, heterocycle, —OR¹², and —CH₂OR¹², wherein R¹² is selected from the group consisting of C₁₋₆alkyl and —C(O)R¹²;
R⁶ is selected from the group consisting of hydrogen, C₆₋₁₄aryl, C₁₋₆alkyl, C₁₋₆alkylheterocycle, heterocycle, —OR¹², and —CH₂OR¹²; and
n=1-4
said process comprising:
exposing a compound having the formula 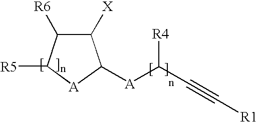
wherein R¹-R⁶ are as hereinbefore defined,
to light with a wavelength of 200 to 400 nanometers in the presence of a compound of formula NR⁷R⁸R⁹, wherein R⁷, R⁸ and R⁹, are independently selected from the group consisting of hydrogen, C₆₋₁₄aryl, C₁₋₆alkyl, C₁₋₆alkylheterocycle, and heterocycle.

2. A process for the preparation of 3-methylenehexahydrofuro[2,3-b]furan in the absence of radical initiators comprising exposing a 3-halo-2-(2-propynyloxy)tetrahydrofuranyl derivative to light with a wavelength from 200 to 400 nanometers, in the presence of a solvent containing a compound of formula NR⁷R⁸R⁹, wherein R⁷, R⁸ and R⁹, are independently selected from the group consisting of hydrogen, C₆₋₁₄aryl, C₁₋₆alkyl, C₁₋₆alkylheterocycle, and heterocycle, thereby cyclizing the 3-halo-2-(2propynyloxy)tetrahydrofuranyl derivative to form 3-methylenehexahydrofuro[2,3-b]furan.

3. A process for the preparation of hexahydrofuro[2,3-b]furan-3-ol consisting of:
a) exposing a 3-halo-2-(2-propynyloxy)tetrahydrofuranyl derivative to light with a wavelength from 200-400 nanometers in the presence of a solvent containing a compound of formula NR⁷R⁸R⁹, wherein R⁷, R⁸ and R⁹, are independently selected from the group consisting of hydrogen, C₆₋₁₄aryl, C₁₋₆alkyl, C₁₋₆alkylheterocycle, and heterocycle, thereby cyclizing the 3-halo-2-(2-propynyloxy)tetrahydrofuranyl derivative to form 3methylenehexahydrofuro[2,3-b]furan;
b) oxidizing said 3-methylenehexahydrofuro[2,3-b]furan to produce tetrahydrofuro[2,3b]furan-3(2M-one; and
c) reducing said tetrahydrofuro[2,3-b]furan-3(2H)-one to yield hexahydrofuro[2,3b]furan-3-ol.

4. A process according to claim 2 or 3, wherein the 3-halo-2-(2propynyloxy)tetrahydrofuranyl derivative is selected from the group consisting of 3-iodo2-(2-propynyloxy)tetrahydrofuran, 3-bromo-2-(2-propynyloxy)tetrahydrofuran, and 3chloro-2-(2-propynyloxy)tetrahydrofuran.

5. A process according to any of claims 1-3 wherein said light is ultraviolet light.

6. A process according to any of claims 1-3 wherein said light is at a wavelength of 254 nanometers.

7. A process according to any of claims 1-3 wherein said compound of formula NR⁷R⁸R⁹ is triethylamine.

8. A process according to claim 2 or 3 wherein said solvent contains water.

9. A process according to claims 2 or 3 wherein said 3-halo-2-(2propynyloxy)tetrahydrofuranyl derivative is selected from the group consisting of 3-iodo2-(2-propynyloxy)tetrahydrofuran. 3-bromo-2-(2-propynyloxy)tetrahydrofuran, and 3chloro-2-(2-propynyloxy)tetrahydrofuran; said light is at a wavelength of 254 nanometers; and said compound of formula NR⁷R⁸R⁹ is triethylamine.

10. A process according to any of claims 1-3 wherein the process is performed in an apparatus suitable for use as a flow-cell reactor.
